(19) United States
(12) Patent Application Publication
(10) Pub. No.: US 2009/0197837 A1
(43) Pub. Date: Aug. 6, 2009

(54) ALENDRONATE FORMULATIONS, METHOD OF MAKING AND METHOD OF USE THEREOF

(76) Inventors: Vivek S. Desai, Ellicott City, MD (US); Yogeeta Suresh Narkar, Pikesville, MD (US)

Correspondence Address: CANTOR COLBURN, LLP, 20 Church Street, 22nd Floor, Hartford, CT, 06103, US

(21) Appl. No.: 12/365,472

(22) Filed: Feb. 4, 2009

Related U.S. Application Data
(60) Provisional application No. 61/026,156, filed on Feb. 5, 2008.

(51) Int. Cl. A61K 31/66; A61P 19/10

(52) U.S. Cl. 514/103

(57) ABSTRACT

Disclosed is a liquid, oral dosage form comprising alendronic acid or pharmaceutically acceptable salts thereof, a process for the preparation of such liquid dosage forms, and use thereof.

### CROSS REFERENCE TO RELATED APPLICATION

This application claims the benefit of U.S. Provisional Application Ser. No. 61/026,156 filed Feb. 5, 2008, which is hereby incorporated by reference in its entirety.

### FIELD OF THE INVENTION

This invention pertains to liquid, oral dosage forms comprising alendronic acid or pharmaceutically acceptable salts thereof, a process for the preparation of such liquid dosage forms, and use thereof.

### BACKGROUND

Bisphosphonate alendronic acid (4-amino-1-hydroxybutylidene-1,1-bisphosphonic acid) and its salts are useful as inhibitors of osteoclast-mediated bone-resorption. These agents can be used in the treatment of bone diseases, particularly for preventing bone resorption in bone diseases such as osteoporosis.

A liquid, oral dosage form of alendronate sodium is known as disclosed in U.S. Pat. No. 5,462,932 to Brenner et al. As disclosed in U.S. Pat. No. 5,462,932, a buffer such as citrate is necessary to maintain the pH of the solution from 2-8. Furthermore, the disclosed formulations require a complexing agent necessary to prevent the formation of insoluble complexes of alendronate to form and precipitate from the aqueous medium: “[a] complexing agent is also present to prevent the precipitation of alendronate through metal complex formation with dissolved metal ions, e.g., Ca, Mg, Fe, Al, Ba, which may leach out of glass containers or rubber stoppers or be present in ordinary tap water. The agent acts as a competitive complexing agent with the alendronate and produces a soluble metal complex whereas alendronate generally forms an insoluble metal complex. Complexing agents include the citrate buffer, which acts as a buffer/complexing agent or EDTA [ethylene diamine tetraacetic acid]. When EDTA is used, it is used in an amount of 0.005-0.1% by weight of the composition and 0.005-2 parts of EDTA to 1 part by weight alendronate and preferably about 0.01% by weight of the composition. Preferred is where citrate buffer is used alone.”

There thus exists a need in the art for other stable liquid, oral dosage forms of alendronate.

### SUMMARY OF THE INVENTION

In one embodiment, a solution composition for oral administration comprises alendronate; deionized water; a sweetener; and a preservative; wherein the pH of the solution composition is about 5.5 to about 7.5; and wherein the solution composition is free of an agent that complexes with multivalent metal ions or is free of a buffering agent.

In another embodiment, a process of preparing a solution composition for oral administration comprises combining alendronate, deionized water, a sweetener, and a preservative to form a mixture; and optionally adjusting the pH of the mixture by adding a pH adjusting agent to form a solution composition having a pH of about 5.5 to about 7.5; and wherein the solution composition is free of an agent that complexes with multivalent metal ions or is free of a buffering agent.

In yet another embodiment, a method of treating osteoporosis in a patient in need thereof comprises administering to the patient a solution composition described herein.

In still yet another embodiment, a method of treating a patient comprises administering a solution composition described herein to a patient in need of alendronate therapy, wherein the composition is administered to treat or prevent osteoporosis in women or men, for the maintenance of bone mass, to reduce the risk of bone fracture, to increase bone mass in men with osteoporosis, to treat glucocorticoid-induced osteoporosis in men or women or bone loss resulting from side effects of other medical treatment, to treat Paget's disease of bone in men or women, to treat bone fractures, osteoarthritis, osteohalisteresis, osteomalacia, bone loss resulting from multiple myeloma and other forms of cancer, and age-related loss of bone mass.

These and other advantages of the invention, as well as additional inventive features, will be apparent from the description of the invention provided herein.

### DETAILED DESCRIPTION OF THE INVENTION

It has been surprisingly found that a stable aqueous solution composition of alendronate can be prepared without the use of a complexing agent or buffer to prevent unwanted alendronate precipitates. The dosage forms disclosed herein are stable aqueous compositions that do not form alendronate precipitates. It has been found that several factors can be controlled to provide a stable aqueous alendronate solution: use of purified (deionized) water in the manufacturing process, adjustment of the pH of the solution to about 5.5 to about 7.5, and minimization of time the composition is exposed to metal-based manufacturing equipment.

The aqueous solution compositions disclosed herein comprise as an active agent alendronic acid or a pharmaceutically acceptable salt thereof. The term “active agent” is meant to include solvates (including hydrates) of the free compound or salt, crystalline and non-crystalline forms, as well as various polymorphs. Unless otherwise specified, the term “active agent” is used herein to indicate alendronic acid or a pharmaceutically acceptable salt thereof. For example, an active agent can include all optical isomers of the compound and pharmaceutically acceptable salts thereof either alone or in combination.

“Pharmaceutically acceptable salt” includes derivatives of the disclosed compounds, wherein the parent compound is modified by making an addition salt thereof, and further refers to pharmaceutically acceptable solvates, including hydrates, of such salts. Examples of pharmaceutically acceptable salts include, but are not limited to, alkali or organic addition salts of acidic residues such as carboxylic acids; and the like. For example, acceptable inorganic salts include metal salts such as sodium salt, potassium salt, cesium salt, and the like; and alkaline earth metal salts, such as calcium salt, magnesium salt, and the like, and a combination comprising one or more of the foregoing salts. Specific salts include alkali metal salts such as potassium and sodium.

As used herein “alendronate” is inclusive of alendronic acid or its pharmaceutically acceptable salt forms. A specific alendronate is the monosodium salt, more specifically 4-amino-1-hydroxybutylidene-1,1-bisphosphonic acid monosodium trihydrate. Methods for preparing alendronate can be found in, for example, U.S. Pat. Nos. 4,922,007 and 5,019,651.

The solution composition can contain an amount of alendronate such that the alendronate is completely solubilized in the aqueous composition. As used herein “completely solubilized” means that no undissolved solids are visually observed in the composition at room temperature.

The amount of alendronate administered to a patient can be calculated by one of ordinary skill in the art knowing the concentration of alendronate in the solution composition. Exemplary doses of alendronate is about 1.5 to 3000 μg/kg of body weight and specifically about 10 to about 200 μg/kg of body weight.

The solution composition further comprises water, a sweetener, a preservative, optional colorant, and optional pH adjusting agent, wherein the solution composition is free of an agent that complexes with multivalent metal ions or is free of a buffering agent.

The water used to prepare the solution compositions is specifically purified water USP. The water can be purified or deionized to remove multi-valent metal cations using purification techniques well-known in the art, for example distillation, ion-exchange, reverse osmosis, and the like.

The solution composition includes a sweetener to make the composition palatable and more pleasing to the patient and to mask the taste of the alendronate. Exemplary sweeteners include sugar alcohols (or polyols), such as glycerol, sorbitol, xylitol, mannitol, galactitol, maltitol, hydrogenated isomaltulose (isomalt), lactitol, erythritol, glucitol, ribitol or a combination comprising at least one of the foregoing; sugar sweeteners generally include saccharides, such as mono-saccharides, di-saccharides and poly-saccharides such as sucrose (sugar), dextrose, maltose, dextrin, maltodextrin, xylose, ribose, glucose (including liquid glucose), mannose, galactose, fructose (levulose), lactose, invert sugar, fructo oligo saccharide syrups, trehalose, tagatose, fucose, gulose, raffinose, ribulose, rufinose, saccharose, stachyose, xylulose, adonose, amylase, arabinose, deoxyribose, corn syrup solids, such as high fructose corn syrup, or a combination comprising at least one of the foregoing; artificial sweeteners such as soluble saccharin salts, i.e., sodium or calcium saccharin salts, the potassium salt of 3,4-dihydro-6-methyl-1,2,3-oxathiazine-4-one-2,2-dioxide (Acesulfame-K), the free acid form of saccharin, L-aspartic acid derived sweeteners, such as L-aspartyl-L-phenylalanine methyl ester (Aspartame), L-alphaaspartyl-N-(2,2,4,4-tetramethyl-3-thietanyl)-D-alaninamide hydrate (Alitame), N—[N-(3,3-dimethylbutyl)-L-aspartyl]-L-phenylalanine 1-methyl ester (Neotame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, or a combination comprising at least one of the foregoing; maltol; or a combination comprising at least one of the foregoing sweeteners.

The sweetener can be present in the solution composition in an amount of about 0.1 to about 75 weight percent based on the total weight of the solution composition, specifically about 5 to about 50 weight percent, and more specifically about 2.5 to about 25 weight percent. The amount of sweetener can be determined by one of ordinary skill in the art without undue experimentation. The use of sensory panels to determine the acceptable sweetness of the solution composition may be used.

In one embodiment, the sweetener is a sorbitol solution present in the solution composition in an amount of about 1 to about 75 weight percent sorbitol solution based on the total weight of the solution composition, specifically about 5 to about 35 weight percent, and more specifically about 5 to about 20 weight percent. The sorbitol solution can be a solution containing a sorbitol solids amount of about 50 to about 80% w/w in water, specifically about 60 to about 70 w/w.

In another embodiment, the sweetener is a maltitol solution present in the solution composition in an amount of about 1 to about 75 weight percent maltitol solution based on the total weight of the solution composition, specifically about 5 to about 35 weight percent, and more specifically about 2.5 to about 20 weight percent. The maltitol solution can be a solution containing a maltitol solids amount of about 5 to about 85% w/w solution in water, specifically 20 to about 75% w/w, more specifically about 40 to about 65 w/w, and yet more specifically about 50 to about 55% w/w.

The solution composition further includes a preservative to prevent the unwanted growth of bacteria, molds, fungi, or yeast. Examples of suitable preservatives include benzoic acid alkali metal salts (e.g., sodium benzoate), sorbic acid alkali metal salts (e.g., potassium sorbate), sodium erythorbate, sodium nitrite, calcium sorbate, butylated hydroxyanisole (BHA), butylated hydroxytoluene (BFT), parabens (e.g., lower alkyl esters of para-hydroxybenzoic acid), alkali metal salts of parabens including sodium and potassium salts of methyl-, ethyl-, propyl-, or butylparaben, or a combination comprising at least one of the foregoing preservatives. Specific preservatives include sodium methylparaben, sodium propylparaben, and sodium butylparaben.

The preservative is present in the solution composition in an amount of about 0.001 to about 0.15 weight percent based on the total weight of the composition, specifically about 0.0075 to about 0.05 weight percent, and yet more specifically about 0.01 to about 0.04 weight percent.

In one embodiment, the preservative is a combination of sodium propylparaben, and sodium butyl paraben in an amount about 0.01 to about 0.1 weight percent sodium propylparaben and about 0.001 to about 0.0075 weight percent sodium butylparaben based on the total weight of the composition.

In another embodiment, the preservative is a combination of sodium methylparaben, sodium propylparaben, and sodium butylparaben in an amount of about 0.01 to about 0.13 weight percent sodium methylparaben, about 0.01 to about 0.1 weight percent sodium propylparaben, and about 0.001 to about 0.0075 weight percent sodium butylparaben based on the total weight of the composition.

The solution composition may further optionally include a pH adjusting agent to render the final solution composition with a pH of about 5.5 to about 7.5. Suitable pH adjusting agents include pharmaceutically acceptable acids, bases, and their salts. Exemplary pH adjusting agents include alkali metal hydroxides (e.g., sodium hydroxide and potassium hydroxide), hydrochloric acid, alkali metal carbonates (e.g., sodium carbonate and potassium carbonate), carbonic acid, or a combination comprising at least one of the foregoing pH adjusting agents. The pH adjusting agents can be used as solutions or suspensions in a pharmaceutically acceptable solvent. Suitable pharmaceutically acceptable solvents for use with the pH adjusting agent can include purified water, lower alkyl alcohols such as ethanol, a glycol, and the like, or a combination comprising at least one of the foregoing solvents.

The amount of pH adjusting agent can be any amount to result in a pH of the final solution composition of about 5.5 to about 7.5, specifically about 6.0 to about 7.3, more specifically about 6.3 to about 7.2, even more specifically about 6.5 to about 7.0, and still yet more specifically about 6.8. Specific amounts of pH adjusting agent can be about 0.001 to about 10 weight percent based on the total weight of the solution composition, more specifically 0.01 to about 5.0 weight percent, and yet more specifically about 0.1 to about 1.0 weight percent.

The solution composition may optionally further comprise a flavoring agent. Flavoring agents include those flavors known to one of ordinary skill in the art, such as natural flavors and artificial flavors. Suitable amounts of flavoring agent can be selected by one of ordinary skill in the art without undue experimentation. In one embodiment, the flavoring agent can be present in the solution composition from about 0.1 to about 8.0 weight percent based on the total weight of the solution composition, specifically about 0.4 to about 6 weight percent, and more specifically about 1.0 to about 3.0 weight percent.

The solution composition may optionally further comprise a colorant conventional in the pharmaceutical art. Colorants can be used in amounts effective to produce a desired color for the composition. The colorants may include pigments, natural food colors and dyes suitable pharmaceutical applications.

The solution composition can further comprise an optional additional solvent. Exemplary additional solvents include glycerin; propylene glycol; a lower polyethylene glycol (e.g., polyethylene glycol 200, polyethylene glycol 300, polyethylene glycol 400, polyethylene glycol 540, polyethylene glycol 600, and the like); ethanol; propylene carbonate; or a combination comprising at least one of the foregoing additional solvents.

These additional solvent can be present in the solution composition in an amount of about 1 to about 50 weight percent based on the total weight of the solution composition, specifically about 2 to about 30 weight percent, more specifically about 3 to about 20 weight percent, and yet more specifically about 5 to about 10 weight percent.

As mentioned above, the solution composition is free of an agent that complexes with multivalent metal ions or the solution composition is free of a buffering agent. Exemplary agents that complexes with multivalent metal ions are citrate and EDTA. The buffering agent is a mixture of a weak acid and a soluble salt thereof, or a monocation or dication salt of a dibasic acid. Exemplary buffering agents are alkali metal citrate, citric acid/sodium citrate, potassium hydrogen tartrate, sodium hydrogen tartrate, potassium hydrogen phthalate, sodium hydrogen phthalate, potassium dihydrogen phosphate, disodium hydrogen phosphate, and the like.

As mentioned, alendronate is known to precipitate in the presence of multivalent metal ions. The solution composition disclosed herein is stable in a stainless steel tank for a period of three days without any observable precipitation. The solution composition is packaged in material substantially free or completely free of multivalent metal ions (e.g., poly(ethylene terephthalate) containers).

To minimize the potential for precipitate formation, the exposure of the solution composition to metal-based manufacturing equipment during its preparation is controlled. Specifically, the solution composition is exposed to metal-based manufacturing equipment for less than about 85 hours, specifically less than about 72 hours, more specifically less than about 60 hours, and yet more specifically less than about 48 hours starting from the time the alendronate is introduced into the manufacturing process to make the solution composition.

The solution composition exhibits physical and chemical stability for extended periods of time. In one embodiment, the solution composition exhibits no precipitation by visual observation when stored at room temperature (about 25° C.) or at refrigerated temperature (about 4 to 8° C.) for a period of nine months, more specifically for a period of twelve months, yet more specifically for a period of twenty-three months. In another embodiment, the solution composition exhibits no precipitation by visual observation when exposed to accelerated aging conditions (temperature 40° C. and 75% relative humidity) for a period of three months.

The presence of precipitates can be determined by visual observation or using techniques well known to one having ordinary skill in the art. Exemplary techniques include visual observation using a light box, laser light scattering liquid particle counters, light obscuration (blocking) liquid particle counters (e.g., liquid particle counters available from HACH ULTRA), and the like.

In one embodiment, the aqueous solution composition is free of a viscosity agent such as carboxymethylcellulose, sodium carboxymethyl cellulose, xanthan gum, microcrystalline cellulose, alginate, propylglycol alginate, Arabic gum (acacia), guar gum, locust bean, carrageenan gum, karaya gum, tragacanth gum, chitosan, carbomer, and the like, and combinations thereof.

The aqueous solution compositions containing alendronate are suitable for oral administration to treat a patient in need of alendronate therapy.

Also included herein are methods of using the solution composition to treat a patient in need of alendronate therapy. Specifically, the solution composition is useful for the treatment or prevention of osteoporosis in women (e.g., postmenopausal women) or men, for the maintenance of bone mass, to reduce the risk of bone fracture, as treatment to increase bone mass in men with osteoporosis, for the treatment of glucocorticoid-induced osteoporosis in men or women or bone loss resulting from side effects of other medical treatment, treatment of Paget's disease of bone in men or women, treating bone fractures, osteoarthritis, osteohalisteresis, osteomalacia, bone loss resulting from multiple myeloma other forms of cancer, and age-related loss of bone mass.

The following examples further illustrate the invention but, of course, should not be construed as in any way limiting its scope.

### EXAMPLES

### Example 1

### Aqueous Solution of Alendronate Sodium, Equivalent 70 Milligram Base Per 75 Milliliters Final Composition

Several 70 mg base equivalent alendronate sodium per 75 ml aqueous solutions are prepared having the following components and amounts shown in Table 1.

TABLE 1

| | Amount | | | | |
|---|---|---|---|---|---|
| Ingredient | A | B | C | D | E |
| Alendronate | 70 milligram | 70 milligram | 70 milligram | 70 milligram | 70 milligram |
| sodium USP | base equivalent | base equivalent | base equivalent | base equivalent | base equivalent |
| | per 75 ml | per 75 ml | per 75 ml | per 75 ml | per 75 ml |
| | composition | composition | composition | composition | composition |
| Purified water | q.s. | q.s. | q.s. | q.s. | q.s. |
| USP | | | | | |
| Maltitol solution | 2.5-7.5 | 3.75-6.25 | — | — | — |
| NF | g/100 ml | g/100 ml | | | |
| Sorbtiol 70% | — | — | 8-12 | 13-17 | 18-22 |
| solution USP | | | ml/100 ml | ml/100 ml | ml/100 ml |
| Saccharin sodium | 0.005-0.015 | 0.0075-0.0125 | 0.005-0.015 | 0.005-0.015 | 0.0075-0.0125 |
| USP | g/100 ml | g/100 ml | g/100 ml | g/100 ml | g/100 ml |
| Sodium | 0.0009-0.0027 | 0.00135-0.00225 | 0.0009-0.0027 | 0.0009-0.0027 | 0.00135-0.00225 |
| hydroxide NF | g/100 ml | g/100 ml | g/100 ml | g/100 ml | g/100 ml |
| Flavor | 0.26-0.78 | 0.39-0.65 | 0.26-0.78 | 0.26-0.78 | 0.39-0.65 |
| | g/100 ml | g/100 ml | g/100 ml | g/100 ml | g/100 ml |
| Sodium | 0.0225-0.0425% | 0.0275-0.0425% | 0.0225-0.0425% | 0.0225-0.0425% | 0.0275-0.0425% |
| propylparaben | | | | | |
| Sodium | 0.005-0.0075% | 0.006-0.0075% | 0.005-0.0075% | 0.005-0.0075% | 0.006-0.0075% |
| butylparaben | | | | | |
| Sodium | * | * | * | * | * |
| hydroxide NF | | | | | |
| Hydrochloric acid | * | * | * | * | * |
| NF | | | | | |

*Added to adjust the pH to 6.6-7.0 if needed

The formulation ingredients are dissolved in water sequentially to obtain a uniform homogenous solution. If necessary, the final pH is adjusted to 6.6-7.0 using dilute solutions of sodium hydroxide NF and/or hydrochloric acid NF.

### Example 2

### Stability Studies of the Aqueous Solution of Alendronate Sodium

Aqueous formulations are exposed to varying temperatures and relative humidity (RH) (25° C./60% RH) over a 1, 2, 3 6, 9, and 12-month timeframe to study the stability of the alendronate in solution. The results are compared to Fosamax® oral solution aged under the same conditions. The results, for active and preservative assay and particulate matter, indicate that the alendronate remains stable under these aging conditions.

Each of the five aqueous solutions formulated with varying amounts of sorbitol or maltitol are stored in USP Type I glass bottles (borosilicate glass) or USP Type III glass bottles (soda lime glass) for extended periods of time and varying temperature. All samples exhibited no precipitation by visual observance after three months at room temperature, after three months under refrigeration, or after three months at 40° C.

An aqueous solution formulated with 5% maltitol and stored in plastic (polyethylene terephthalate, “PET”) bottles exhibited no precipitation by visual observance after twenty-three months at room temperature, after twenty-three months under refrigeration, or after three months at 40° C.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. “Or” means and/or. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A solution composition for oral administration, comprising:
alendronate;
deionized water;
a sweetener; and
a preservative;
wherein the pH of the solution composition is about 5.5 to about 7.5; and
wherein the solution composition is free of an agent that complexes with multivalent metal ions or is free of a buffering agent.

2. The solution composition of claim 1, wherein the solution composition has a pH of about 6.0 to about 7.0.

3. The solution composition of claim 1, wherein the solution composition has a pH of about 6.8.

4. The solution composition of claim 1, wherein the alendronate is alendronate sodium.

5. The solution composition of claim 1, wherein the sweetener is a sugar alcohol, glycerol, sorbitol, xylitol, mannitol, galactitol, maltitol, hydrogenated isomaltulose, lactitol, erythritol, glucitol, ribitol, a saccharide, a mono-saccharide, a di-saccharide, a poly-saccharide, sucrose, dextrose, maltose, dextrin, maltodextrin, xylose, ribose, glucose, mannose, galactose, fructose, lactose, invert sugar, a fructo oligo saccharide syrup, trehalose, tagatose, fucose, gulose, raffinose, ribulose, rufinose, saccharose, stachyose, xylulose, adonose, amylase, arabinose, deoxyribose, a corn syrup solid, high fructose corn syrup, an artificial sweetener, saccharin sodium, calcium saccharin, 3,4-dihydro-6-methyl-1,2,3-oxathiazine-4-one-2,2-dioxide potassium salt (Acesulfame-K), saccharin free acid, a L-aspartic acid derived sweetener, L-aspartyl-L-phenylalanine methyl ester (Aspartame), L-alphaaspartyl-N-(2,2,4,4-tetramethyl-3-thietanyl)-D-alaninamide hydrate (Alitame), N—[N-(3,3-dimethylbutyl)-L-aspartyl]-L-phenylalanine 1-methyl ester (Neotame), methyl esters of L-aspartyl-L-phenylglycerine, L-aspartyl-L-2,5-dihydrophenyl-glycine, L-aspartyl-2,5-dihydro-L-phenylalanine, L-aspartyl-L-(1-cyclohexen)-alanine, maltol, or a combination comprising at least one of the foregoing sweeteners.

6. The solution composition of claim 1, wherein the sweetener is present in the composition at about 0.1 to about 75 weight percent based on the total weight of the solution composition.

7. The solution composition of claim 1, wherein the preservative is a benzoic acid alkali metal salt, sodium benzoate, a sorbic acid alkali metal salt, potassium sorbate, sodium erythorbate, sodium nitrite, calcium sorbate, butylated hydroxyanisole, butylated hydroxytoluene, a paraben, an alkali metal salt of a paraben, sodium methylparaben, sodium propylparaben, sodium butylparaben, or a combination comprising at least one of the foregoing preservatives.

8. The solution composition of claim 1, wherein the preservative is present in the composition at about 0.001 to about 0.15 weight percent based on the total weight of the composition.

9. The solution composition of claim 1, further comprising a pH adjusting agent.

10. The solution composition of claim 1, comprising:
alendronate sodium;
saccharin sodium;
maltitol or sorbitol;
sodium propylparaben; and
sodium butylparaben.

11. The solution composition of claim 10, comprising:
about 0.0075 to about 0.0125 g saccharin sodium per 100 ml of the solution composition;
about 3.75 to about 6.25 g maltitol solution NF per 100 ml of the solution composition;
about 0.0225 to about 0.0425 percent sodium propylparaben; and
about 0.006 to about 0.0075 percent sodium butylparaben.

12. The solution composition of claim 10, comprising:
about 0.005 to about 0.015 g saccharin sodium per 100 ml of the solution composition;
about 10 to about 20 ml 70% sorbitol solution per 100 ml of the solution composition;
about 0.0225 to about 0.0425 percent sodium propylparaben; and
about 0.005 to about 0.0075 percent sodium butylparaben.

13. The solution composition of claim 1, wherein no precipitation is visually observed in the composition after twenty-three months at 25° C.

14. The solution composition of claim 1, wherein no precipitation is visually observed in the composition after three months at 40° C.

15. The solution composition of claim 1 free of a viscosity agent.

16. A method of preparing a solution composition for oral administration, comprising:
combining alendronate, deionized water, a sweetener, and a preservative to form a mixture; and
optionally adjusting the pH of the mixture by adding a pH adjusting agent to form a solution composition having a pH of about 5.5 to about 7.5;
and
wherein the solution composition is free of an agent that complexes with multivalent metal ions or is free of a buffering agent.

17. The method of claim 16, wherein the solution composition has a pH of about 6.0 to about 7.0.

18. The method of claim 16, wherein the solution composition has a pH of about 6.8.

19. A method of treating osteoporosis in a patient in need thereof, comprising administering to the patient the solution composition of claim 1.

20. A method of treating a patient, comprising
administering the solution composition of claim 1 to a patient in need of alendronate therapy, wherein the composition is administered to treat or prevent osteoporosis in women or men, for the maintenance of bone mass, to reduce the risk of bone fracture, to increase bone mass in men with osteoporosis, to treat glucocorticoid-induced osteoporosis in men or women or bone loss resulting from side effects of other medical treatment, to treat Paget's disease of bone in men or women, to treat bone fractures, osteoarthritis, osteohalisteresis, osteomalacia, bone loss resulting from multiple myeloma and other forms of cancer, and age-related loss of bone mass.
